(19) United States
(12) Patent Application Publication
(10) Pub. No.: US 2017/0020751 A1
(43) Pub. Date: Jan. 26, 2017

(54) Animal First Aid Kit

(71) Applicant: Lynne Kunitz, Issaquah, WA (US)

(72) Inventor: Lynne Kunitz, Issaquah, WA (US)

(21) Appl. No.: 15/173,266

(22) Filed: Jun. 3, 2016

Related U.S. Application Data
(63) Continuation-in-part of application No. 14/157,208, filed on Jan. 16, 2014, now abandoned.

(51) Int. Cl. A61F 17/00; A01K 13/00

(52) U.S. Cl. CPC A61F 17/00; A01K 13/00

(57) ABSTRACT

The instant application discloses, among other things, an animal first aid kit. The animal first aid kit may comprise any container capable of holding its contents. The contents may include medicines and supplies that allow the user to evaluate an animal or to tend to it by providing comfort, controlling bleeding, or preventing further injury or infection, for example, as well as prescription medicine obtained from a veterinary. The animal first aid kit may also include a pamphlet describing its contents and providing instructions on its use. In one embodiment, the pamphlet may direct the user to obtain a prescription for a particular device or medicine, for example, for an antihistamine such as Diphenhydramine.

### FIELD

This disclosure generally relates to an Animal First Aid Kit.

### BACKGROUND

Animals may sustain a variety of injuries, accidents, and health conditions during their lifespan, with the majority occurring outside the home. People rarely have supplies to apply first aid animals, particularly when hiking.

### SUMMARY

An Animal First Aid Kit containing a variety of components that may provide a convenient way for a person to assist an injured or sick animal. Owners or caretakers may also call an animal emergency clinic to be directed to use the supplies available in the kit. It is not meant to replace proper veterinary care, but to allow first aid to be administered until proper care is obtained.

The instant application discloses, among other things, an Animal First Aid Kit, which may comprise a variety of components. The Animal First Aid Kit may be configured to allow a user to treat a variety of domesticated pets, or it may be tailored for a specific type of animal, for example, canines. The Animal First Aid Kit may include medical supplies such as, but not limited to, triple antibiotic ointment, hydrogen peroxide tablets, electrolyte tablets, saline solution, bandages, gauze, dressings, styptic powder, cotton swabs, ear cleaner, gloves, scissors, and forceps. The Animal First Aid Kit may also include an instructional pamphlet describing the contents. The pamphlet may include instructions directing a user to obtain a prescription for or purchase over-the-counter a particular medical device or medicine, for example, an antihistamine.

An Animal First Aid Kit may be small and packable, and may be configured with a means of attachment so that it may be easy to carry when doing various activities, such as walking, hiking, camping, or boating.

A person having skill in the art will recognize that Animal First Aid Kit may be comprised of various components. It may also be made of different materials, and the container may be structured to have different shapes, sizes, and colors.

### BRIEF DESCRIPTION OF THE DRAWINGS

FIG. 1 is a perspective view of Animal First Aid Kit, according to one embodiment.

FIG. 2 is an interior view of Animal First Aid Kit, according to one embodiment.

FIG. 3 is a perspective view of Animal First Aid Kit, according to another embodiment.

FIG. 4 is a bottom view of Animal First Aid Kit, according to one embodiment.

FIG. 5 is an example of Animal First Aid Kit instructional booklet, according to one embodiment.

Like reference numerals are used to designate like parts in the accompanying drawings.

### DETAILED DESCRIPTION

A more particular description of certain embodiments of Animal First Aid Kit may be had by references to the embodiments shown in the drawings that form a part of this specification, in which like numerals represent like objects.

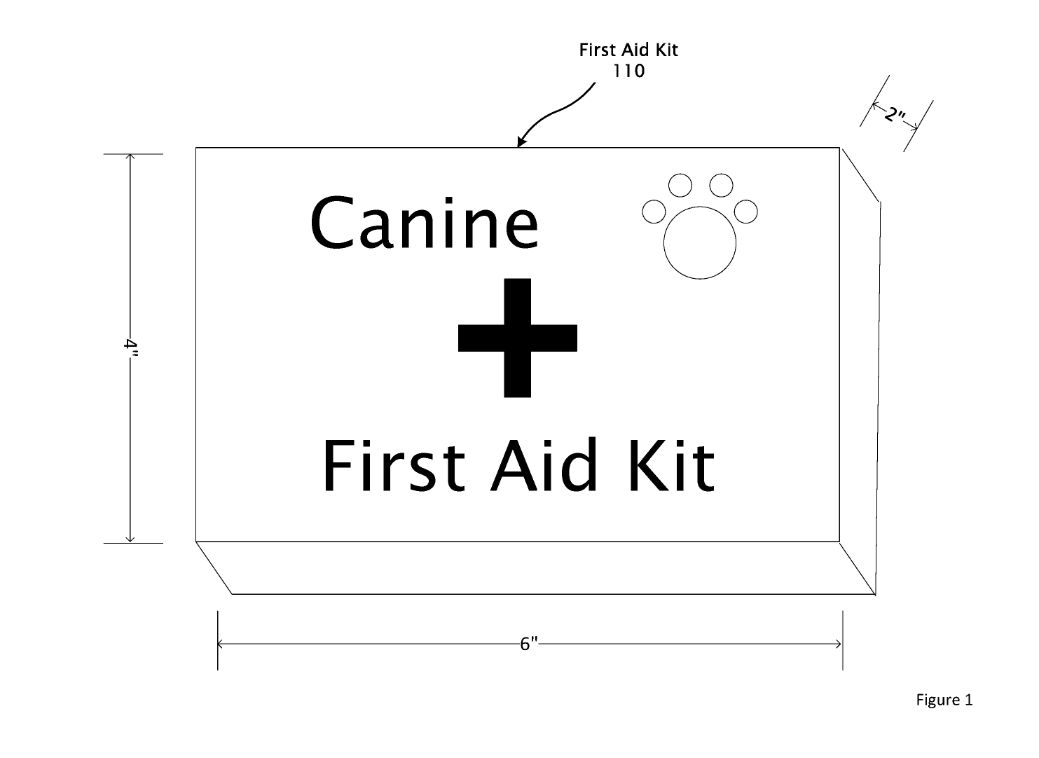

FIG. 1 is a perspective view of Animal First Aid Kit 110, according to one embodiment. Animal First Aid Kit 110 may comprise a container, such as a box, bag, or envelope, for example. The container may be made of any material including, but not limited to, plastic, carbon fiber, nylon, cotton, hemp, metal, silicone, rubber, or wood.

In one embodiment, the Animal First Aid Kit may be made of a material and structure designed to withstand various environmental conditions such changes in temperature, humidity, altitude, or movement. The Animal First Aid Kit may also be made of a material or structure configured in ways that facilitate transportability and storage; for example, the Animal First Aid Kit may include handles, straps, or wheels, or it may have the ability to expand or contract in size.

In this example, a kit configured for use with dogs is displayed. This example of a container is six inches long, four inches wide, and two inches deep. A smaller or larger container may be used. Small sizes, up to eight inches long by six inches in width by three inches in depth, may be more suitable for packing. Such a container may fit in a backpack or suitcase, for example.

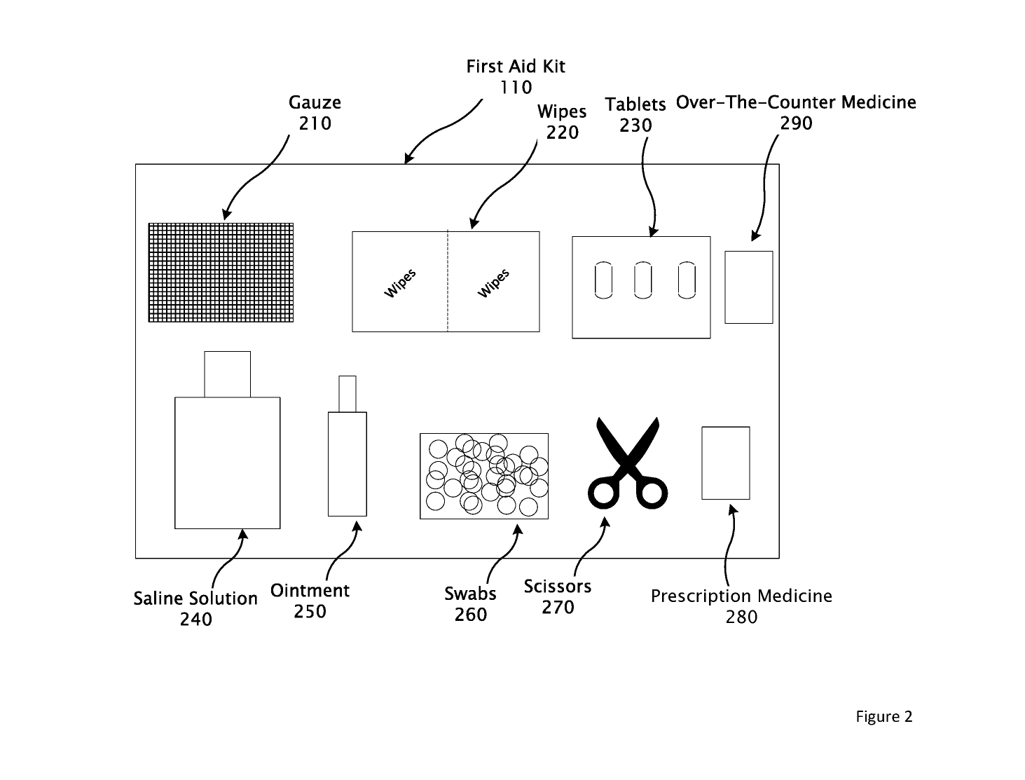

FIG. 2 is an interior view of Animal First Aid Kit 110, according to one embodiment. Animal First Aid Kit 110 may allow a user to treat any domesticated pet, or it may be configured for a specific type of animals, for example, dogs. A person skilled in the art will understand that Animal First Aid Kit 110 may include various types of medicines, supplies, and tools that may enable the user to tend to the animal by providing comfort, controlling bleeding, or preventing infection. Animal First Aid Kit 110 contents may include, but are not limited to, triple antibiotic ointment, hydrogen peroxide tablets, electrolyte tablets, saline solution, bandages, gauze, dressings, styptic powder, cotton swabs, ear cleaner, gloves, scissors, and forceps, over-the-counter medicine, and prescribed medicine. In the example shown, Animal First Aid Kit may contain Gauze 210, Antiseptic Wipes 220, Hydrogen Peroxide Tablets 230, Saline Solution 240, Triple Antibiotic Ointment 250, Cotton Swabs 260, Scissors 270, Prescription Medicine 280, and Over-the-Counter Medicine 290.

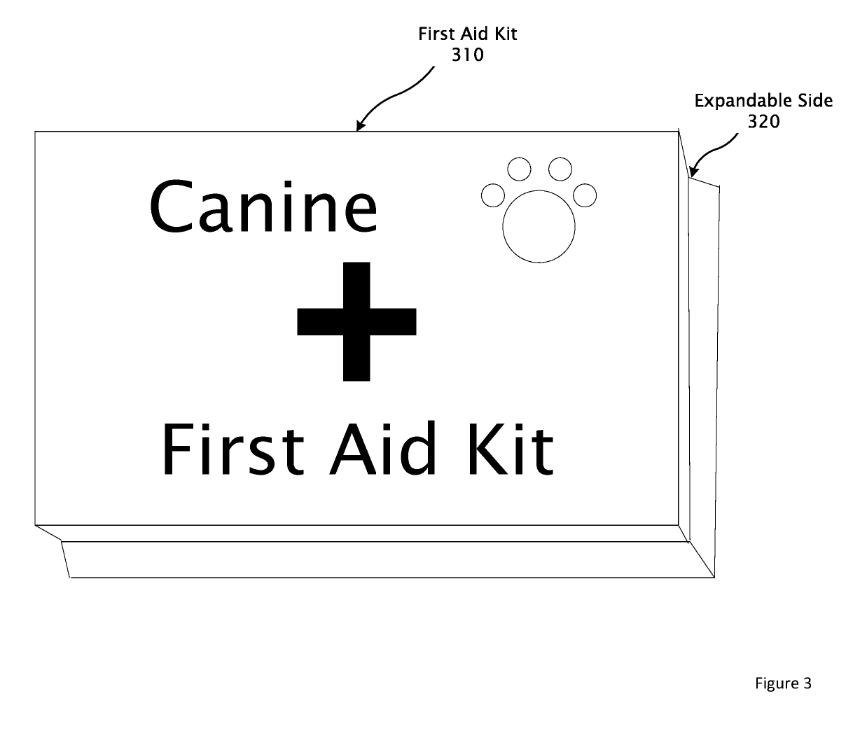

FIG. 3 is a perspective view of Animal First Aid Kit 310, according to another embodiment. in this example, Expandable Side 320 may allow Animal First Aid Kit 310 to grow or decrease in size, depending on the contents. Animal First Aid Kit 310 may be made of a hard material, such as plastic or metal, in which case Expandable Side 320 may act as a hinge. In another embodiment, Animal First Aid Kit 310 may be made of a flexible material, such as nylon or canvas. Expandable Side 320 may involve a folding of material, or may have a material that stretches. In another embodiment, a strap, a spring, or other adjustable means may be used to adjust a size of Animal First Aid Kit 310. Animal First Aid Kit 310 may adjust in any direction, for example, height, width, or length.

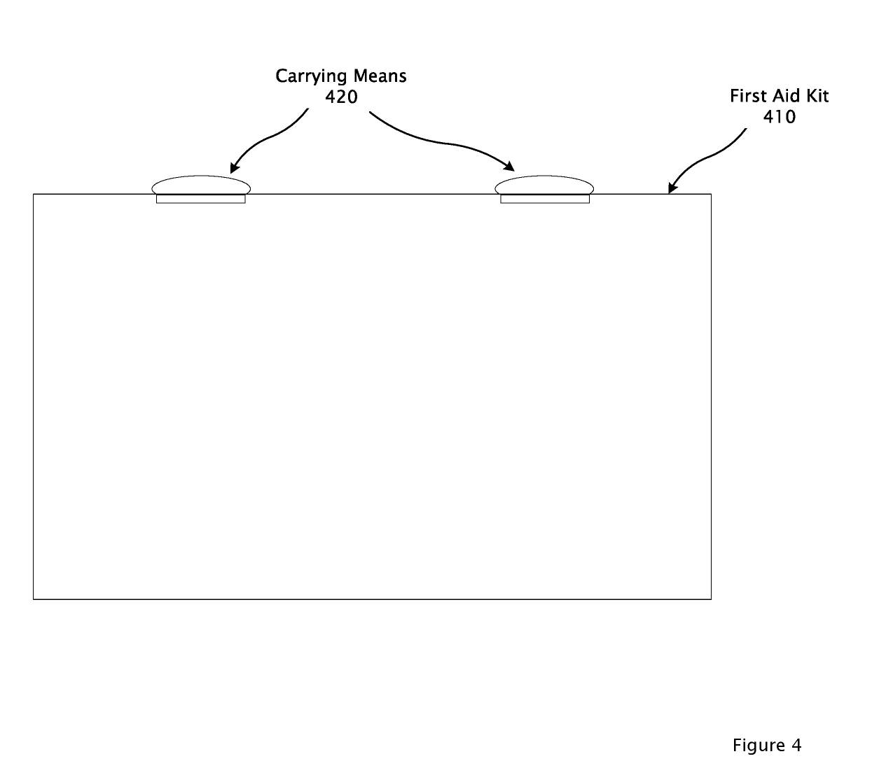

FIG. 4 is a bottom view of Animal First Aid Kit 410, according to one embodiment. Animal First Aid Kit 410 may have Carrying Means 420, which may, for example, provide a simple way to attach Animal First Aid Kit 410 to a backpack, to clothing, or may provide a handle to simplify carrying by hand. Carrying Means 420 may be, for example, a handle, loops, snaps, straps, or hook and loop fasteners. In another embodiment, Carrying Means 420 may be configured to be carried by an animal.

Animal First Aid Kit may also include an instructional pamphlet, providing instructions on how to use one or more of the contents, or direct the user to obtain other supplies.

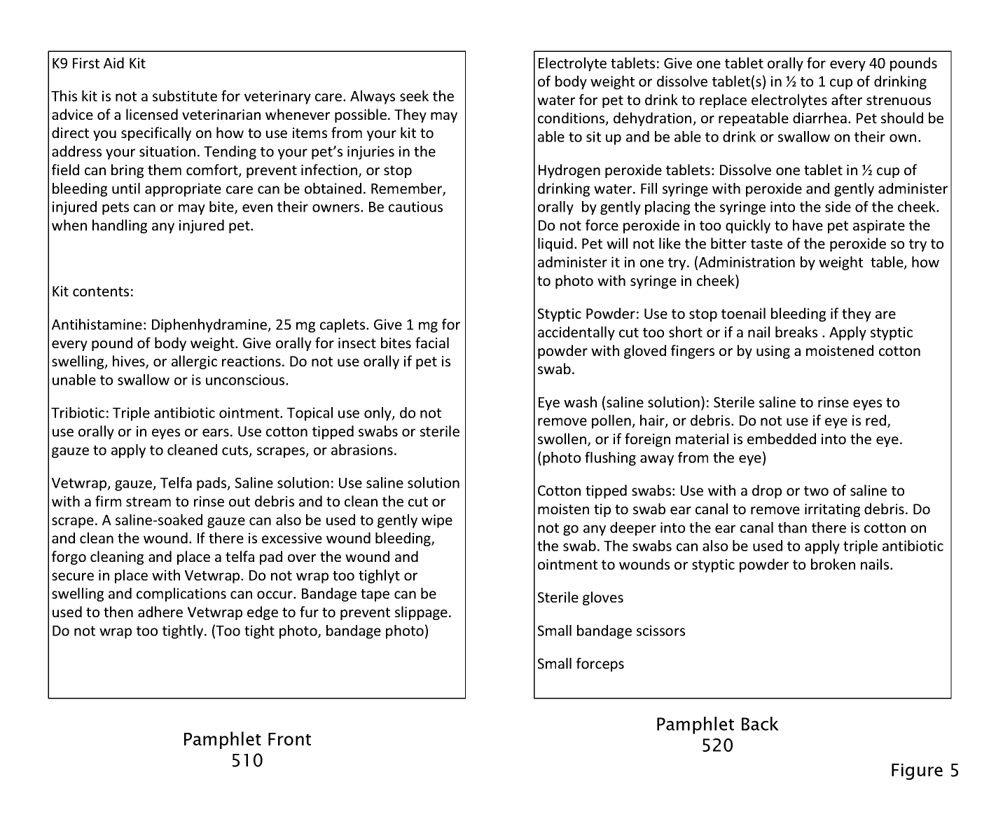

FIG. 5 is an example of an Instructional Pamphlet 510, 520, according to one embodiment. Instructional Pamphlet 510, 520 may describe the contents of the Animal First Aid Kit and provide instructions for its use. Instructional Pamphlet 510, 520 may also direct the user to obtain a prescription from a licensed professional, such as a veterinarian, for a particular type of device or an over-the-counter medicine, for example, for an antihistamine. An injured animal may require various forms of medication and or prescription medicine. Moreover, Instructional Pamphlet 510, 520 may be updated to keep up with the development of new prescription medications and therefore the list of prescription medicines may change over time. Instructional Pamphlet 510, 520 may also contain images, for example, photos of proper ways to bandage or treat particular injuries. Instructional Pamphlet 510, 520 may be laminated and double sided, or it may be a booklet several pages long.

One having skill in the art will recognize that many different designs of Animal First Aid Kit may fall within the spirit and scope of this disclosure, including, for example, varied contents and instructions.

While the detailed description above has been expressed in terms of specific examples, those skilled in the art will appreciate that many other configurations could be used. Accordingly, it will be appreciated that various equivalent modifications of the above-described embodiments may be made without departing from the spirit and scope of the invention.

The foregoing description of various embodiments of the invention has been presented for the purposes of illustration and description. It is not intended to be exhaustive or to limit the invention to the precise form disclosed. It is intended that the scope of the invention be limited not by this detailed description, but rather by the claims appended hereto. The above specification, examples and data provide a complete description of the manufacture and use of the invention. Since many embodiments of the invention can be made without departing from the spirit and scope of the invention, the invention resides in the claims hereinafter appended.

1. A first aid kit, comprising:
supplies for evaluating and treating an animal, including at least one prescription medicine for the animal;
a container to hold the supplies; and
an instructional pamphlet.

2. The first aid kit of claim 1, wherein the container has dimensions no larger than eight inches long by six inches wide by three inches tall.

3. The first aid kit of claim 1, wherein the container is made of one or more materials selected from the group containing plastic, carbon fiber, nylon, cotton, hemp, metal, silicone, rubber, or wood.

4. The first aid kit of claim 1, further comprising a carrying means enabling the kit to be secured to a person, animal, or object.

5. The first aid kit of claim 1, wherein the container is configured to expand or contract in size.

6. The first aid kit of claim 1, further comprising an over-the-counter drug for the animal obtained from a veterinarian.
